(19) United States
(12) Patent Application Publication
(10) Pub. No.: US 2026/0014195 A1
(43) Pub. Date: Jan. 15, 2026

(54) USE OF POLYMER DOTS FOR TREATING SKIN

(71) Applicant: China Medical University, Taichung City (TW)

(72) Inventors: Tzong-Yuan Juang, Taichung City (TW); Chang-Cheng Chang, Taichung City (TW); Hoi-Man Iao, Sec Pai Van (MO); Ching-Hsuan Lin, Taichung City (TW)

(73) Assignee: China Medical University, Taichung City (TW)

(21) Appl. No.: 19/268,976

(22) Filed: Jul. 14, 2025

(30) Foreign Application Priority Data
Jul. 12, 2024 (TW) 113126173

(51) Int. Cl. A61K 31/785; A61N 5/06; A61N 5/067; A61P 17/02

(52) U.S. Cl. CPC A61K 31/785; A61N 5/062; A61N 5/067; A61P 17/02

(57) ABSTRACT

Local tissue defects, liquefaction, infection, and necrosis of skin resulted from various reasons may lead to skin damage and vascular regeneration disorders. Wound hypoperfusion, metabolic disorders, and delayed epithelial cell formation disrupt the process of normal wound healing, thereby leading to the formation of chronic ulcer. To solve the aforementioned problems, a use of polymer dot is provided. The polymer dot may be used for manufacture of a component for the treatment of skin to a subject in need, in order to restore skin damage of the subject, minimize a scar on the damaged skin of the subject, and enhance recovery of the skin of the subject after cosmetic or dermatologic surgery. The polymer dot includes a carbon dot.

### BACKGROUND OF THE INVENTION

### 1. Field of the Invention

The present disclosure relates to the use of polymer dots, more particularly to the polymer dots for skin treatment.

### 2. Description of the Prior Art

Skin is the largest organ of the human body. It serves as a barrier separating the internal body from the external environment, regulates temperature, and provides protection against external stimuli. Maintaining the structural integrity of skin is therefore crucial to ensure its regenerative and reparative capabilities following external trauma or damage.

Typically, when skin undergoes external trauma such as injuries or burns, wounds heal spontaneously. However, some individuals experience a stalled wound healing process due to impaired cellular functions, reduced growth factor levels, and increased pro-inflammatory cytokine levels, leading to chronic wounds and skin infections. Pain and psychological stress resulting from these conditions adversely affect quality of life.

Current approaches to facilitate skin wound healing or repair include the administration of growth factors, gene therapy, cell therapy, and/or skin grafting. However, these methods face issues such as high costs, instability of therapeutic agents or donor tissues, immune rejection, and secondary injury to the wounded area.

Additionally, various nanoparticles (hereinafter referred as NPs) have been employed as drug carriers to promote skin wound healing or repair. However, current NP-based drug delivery systems may cause particle aggregation or undesirable changes in the pharmacological properties of the drugs. Some NPs have also been reported to exhibit cytotoxic effects. For example, metallic silver nanoparticles exhibit cytotoxicity through the release of Ag ions, which readily bind to sulfur-or phosphorus-containing biomolecules, causing cellular damage. Iron oxide nanoparticles can induce inflammation in the endothelial system and provoke cellular oxidative stress, leading to organ dysfunction; such effects are associated with reactive oxygen species (hereinafter referred as ROS) generation induced by nanoparticles.

Therefore, there remains an urgent need in the art to provide improved skin treatment technologies capable of addressing the above-mentioned issues.

### SUMMARY OF THE INVENTION

To overcome the aforementioned problems, the present disclosure provides a method of treating skin, which comprises administering to a subject in need thereof a composition, wherein the composition comprises a polymer dot.

The present disclosure further provides a method for treating skin, which comprises administering to a subject in need thereof a composition and a picosecond laser, wherein the composition comprises a polymer dot.

The present disclosure further provides use of a polymer dot for manufacture of a composition for treating skin of a subject in need, wherein the polymer dot comprises a carbon dot and the composition is configured to promote skin repair and/or skin regeneration of the subject, or enhance wound healing and/or inhibit scar formation on the skin of the subject.

In summary, the polymer dots of the present disclosure address the problems associated with skin wound repair and/or healing identified in the prior art, and provide, in certain embodiments, the following functions and advantages:
(1) Polymer dots disclosed herein possess abundant surface functional groups, excellent biocompatibility, and ease of application.
(2) Polymer dots disclosed herein effectively scavenge free radicals and peroxides.
(3) Polymer dots disclosed herein induce epithelial-mesenchymal interactions (hereinafter referred as EMI) and accelerate collagen remodeling, thereby promoting wound repair and/or regeneration.
(4) Polymer dots disclosed herein, when combined with a picosecond laser treatment, exhibit synergistic effects that accelerate wound repair and healing. Specifically, it effectively shortens the inflammatory phase and promotes epithelial cell migration during the early stage; enhance vascular regeneration and cellular remodeling during the intermediate stage; and effectively control scar formation post-wound closure.
(5) Polymer dots disclosed herein may optionally be combined with other therapeutic drugs or biological agents as needed.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A is a schematic diagram illustrating a preparation process of dendritic polymeric carbon dots according to one embodiment of the present disclosure.

FIG. 1B is a schematic diagram illustrating another preparation process of dendritic polymeric carbon dots according to one embodiment of the present disclosure.

FIG. 2 is a schematic diagram illustrating yet another preparation process of dendritic polymeric carbon dots according to one embodiment of the present disclosure.

FIG. 3A and FIG. 3B are graphs showing the particle size distribution of dendritic polymeric carbon dots according to one embodiment of the present disclosure.

FIG. 3C and FIG. 3D are images showing the morphology of dendritic polymeric carbon dots according to one embodiment of the present disclosure.

FIG. 4 is a schematic diagram illustrating a process for treating skin wounds according to one embodiment of the present disclosure.

FIG. 5 is a schematic diagram illustrating another process for treating skin wounds according to one embodiment of the present disclosure, wherein “Laser 500 pulses” indicates the application of 500 pulses of picosecond laser.

FIG. 6A is an image showing laser-induced optical breakdown at day 0 after skin wound creation and treatment with a picosecond laser according to one embodiment of the present disclosure.

FIG. 6B is an image showing laser-induced optical breakdown at day 3 after skin wound creation and treatment with a picosecond laser according to one embodiment of the present disclosure.

FIG. 7 is an image showing wound healing results from day 0 to day 18 after skin wound creation under different treatment conditions according to one embodiment of the present disclosure.

FIG. 8A is a bar chart showing wound healing results from day 0 to day 9 after skin wound creation under different treatment conditions according to one embodiment of the present disclosure.

FIG. 8B is a line graph showing wound healing results from day 0 to day 9 after skin wound creation under different treatment conditions according to one embodiment of the present disclosure.

FIG. 9 is an image showing immunohistochemical staining results at day 3 after skin wound creation under different treatment conditions according to one embodiment of the present disclosure.

FIG. 10 is an image showing immunohistochemical staining results at day 6 after skin wound creation under different treatment conditions according to one embodiment of the present disclosure.

FIG. 11 is an image showing Masson's trichrome staining results at day 18 after skin wound creation under different treatment conditions according to one embodiment of the present disclosure.

FIG. 12A, FIG. 12B, and FIG. 12C are a bar chart showing ELISA quantitative analysis results at day 18 after skin wound creation under different treatment conditions according to one embodiment of the present disclosure.

FIG. 13 is a bar chart showing additional ELISA quantitative analysis results at day 18 after skin wound creation under different treatment conditions according to one embodiment of the present disclosure.

### DETAILED DESCRIPTION

The following provides a description of embodiments of the present disclosure with reference to specific examples. A person having ordinary skill in the art can readily understand the spirit, advantages, and effects of the present disclosure based on the content herein. However, the described embodiments are provided only for illustrative purposes and are not intended to limit the scope of the present disclosure. The present disclosure may also be implemented in various other forms, and details described herein may be modified or altered without departing from the scope and spirit of the present disclosure.

The proportions, structures, sizes, and other characteristics illustrated in the accompanying drawings are provided solely to facilitate understanding of the present disclosure by a person having ordinary skill in the art. These characteristics are not intended to limit the scope of the present disclosure. Thus, any alterations to proportions, modifications of structures, or adjustments to sizes that do not affect the objectives and effects achievable by the present disclosure shall fall within the scope of the disclosure.

As used herein, the terms “comprises,” “comprising,” and “having,” when referring to specific elements, are intended to indicate the presence of the stated elements, components, structures, steps, or connections, but do not exclude the presence of other elements, components, structures, steps, or connections, unless otherwise explicitly indicated.

Unless explicitly stated otherwise, the singular forms “a,” “an,” and “the” as used herein also include their plural forms. Furthermore, the term “or” as used herein may be interchangeably interpreted with “and/or.”

As used herein, the term “about” refers to a typical tolerance range recognized in the relevant art. For instance, “about” may be interpreted as within approximately two standard deviations of a mean value. When “about” precedes a numerical series or range, it is understood to apply to each number within that series or range. Numeric values herein are intended to encompass variations of ±25%, ±20%, ±10%, ±5%, ±1%, ±0.5%, or ±0.1%. Such variations may result from experimental errors, typical measurement deviations during preparation or processing of compounds, compositions, concentrates, or formulations, variations in starting materials, purity, or manufacturing conditions, among other considerations.

The numeric ranges described herein are inclusive and combinable. Any numeric value within a stated range may serve as an upper or lower boundary to define sub-ranges. For example, a numeric range of “3 nm to 5 nm” encompasses sub-ranges within the stated endpoints, such as 3 nm to 4.5 nm, 3.5 nm to 4 nm, and 4.2 nm to 4.8 nm. Moreover, multiple numeric endpoints described herein may be selected in any combination to define additional numeric ranges; for instance, values such as 3 nm, 4.2 nm, and 5 nm may define ranges including 3 nm to 4.2 nm, 4.2 nm to 5 nm, or 3 nm to 5 nm.

Unless explicitly indicated otherwise, the term “nanoparticles” as used herein is interchangeable with terms such as “nanocarriers,” “nanodrug carriers,” “polymer dots,” “dendritic polymeric carbon dots,” “polymeric carbon dots,” “carbon dots,” “polymeric nano carbon dots,” or “dressings.”

Unless explicitly indicated otherwise, the term “nude mouse” as used herein is interchangeable with the term “mouse.”

Unless explicitly indicated otherwise, the term “hyperbranched” as used herein is interchangeable with the term “dendritic.”

As used herein, the term “polymer dots (hereinafter referred as PDs)” refers to carbon dots having a non-conjugated structure.

As used herein, the term “picosecond laser” refers to an optical physical stimulus capable of inducing laser-induced optical breakdown (hereinafter referred as LIOB). In certain embodiments, picosecond lasers may be employed for the treatment or improvement of scars, pigmentation, facial wrinkles, and/or melasma. High temperatures and pressures generated by the laser are accompanied by plasma expansion and shock wave propagation, thereby enhancing photomechanical effects and reducing thermal damage to surrounding tissues. Induced optical breakdown creates vacuoles within the dermal layer that subsequently collapse, generating localized mechanical forces. These vacuolated regions heal gradually, leading to new collagen formation, attraction of fibroblasts, and enhanced collagen deposition at wound sites, thus stimulating new tissue growth, dermal remodeling, angiogenesis, and re-epithelialization. Additionally, the pressure waves temporarily enhance cell membrane permeability, modify cellular signaling pathways, and initiate cytokine cascades that help improve skin-related conditions. Therefore, compared to conventional laser treatments, picosecond laser exposure duration on the skin is extremely short, significantly reducing thermal damage, minimizing inflammation, and preventing epidermal injury.

As used herein, the term “optical coherence tomography (hereinafter referred as OCT)” (OPXION Technology Inc., Taiwan) refers to a non-invasive imaging technique that acquires real-time, high-resolution three-dimensional images of skin structures and microvasculature through light scattering. OCT is suitable for assisting in skin diagnosis.

As used herein, the term “enzyme-linked immunosorbent assay (hereinafter referred as ELISA)” refers to an analytical method employing specific antibodies for highly selective recognition and binding of target molecules. An enzymatic reaction produces a measurable color change or fluorescent signal, which is quantified using optical instruments. By comparing the optical density or fluorescence intensity of samples against a known standard curve, the concentration of target molecules in the sample is quantitatively determined. ELISA exhibits high sensitivity, allowing precise quantitative analysis of target molecules.

As used herein, the term “immunohistochemistry (hereinafter referred as IHC)” (Quantum Biotechnology Co., Ltd.) refers to a technique using antibodies to recognize and bind specific target antigens. Labeled reagents, such as enzymes or fluorescent dyes, are conjugated to the antibodies, forming complexes that generate staining reactions or fluorescent signals. These signals are visualized using fluorescence microscopy, enabling quantitative evaluation of target antigen (protein) distribution and expression levels within tissues.

As used herein, the term “matrix metalloproteinases (hereinafter referred as MMPs)” refers to a family of calcium-dependent and zinc-containing enzymes primarily involved in cell differentiation, proliferation, and degradation of extracellular matrix (hereinafter referred as ECM). Following skin injury, MMPs participate in ECM degradation and cell migration, essential for regulating inflammation and re-epithelialization of wounds. Appropriate MMP facilitates tissue repair; however, excessive MMP levels can disrupt normal wound healing processes, leading to chronic wounds. Specific MMPs appear at particular locations and stages of wound healing. For example, elevated MMP-2 expression promotes epithelial cell and fibroblast migration into the wound area, while MMP-9 plays a crucial role in keratinocyte migration. Additionally, MMP-2 and MMP-9 regulate angiogenesis factors during wound healing, including tumor necrosis factor-alpha (hereinafter referred as TNF-α) and vascular endothelial growth factor (hereinafter referred as VEGF).

As used herein, the term “epithelial-mesenchymal transition (hereinafter referred as EMT)” refers to a biological process occurring during cellular development. EMT can be classified into three types: type I EMT is associated with embryonic organ development; type II EMT relates to tissue regeneration and organ fibrosis during wound healing; and type III EMT is linked to cancer progression and genetic conditions. In the context of wound healing, type II EMT enables critical tissue repair processes, wherein epidermal cells at wound margins adopt mesenchymal-like features, including altered cell morphology and reduced cell-cell adhesion, concurrently acquiring mesenchymal properties such as increased motility. The expression levels of mesenchymal markers (e.g., fibronectin and vimentin) increase, whereas epithelial marker expression (e.g., E-cadherin) decreases.

As used herein, the term “mitogen-activated protein kinases (hereinafter referred as MAPKs)” refers to a signal transduction pathway activated upon cellular stimulation. Three structurally related yet functionally distinct MAPK pathways exist in mammalian cells, namely ERK1/2, JNK, and p38, which are regulated via phosphorylation cascades. MAPKs guide cellular responses to various stimuli, including inflammatory reactions. The phosphorylation of p38 and JNK is associated with cell proliferation and migration. Transforming growth factor-beta (hereinafter referred as TGF-β) is a crucial growth factor involved in the regulation of various events during wound healing. In the absence of TGF-β signaling, activation of p38 MAPK is delayed, thereby inhibiting cell proliferation in the early stages of wound healing. Thus, both TGF-β and MAPK signaling pathways play vital roles in regulating wound repair processes.

As used herein, the term “transforming growth factor-beta (hereinafter referred as TGF-β)” refers to a multifunctional growth factor secreted by multiple tissues. To date, 33 TGF-β-related genes have been identified in mammalian genomes, implicated in development, physiology, and various diseases. TGF-β regulates wound repair and healing by modulating cellular proliferation and differentiation, extracellular matrix (hereinafter referred as ECM) production, angiogenesis, wound healing, immune modulation, and thereby regulate wound repair or healing. TGF-β signaling is initiated when TGF-β binds to its receptors on the cell surface, activating receptor complexes. Signal transduction occurs through two primary mechanisms: the phosphorylation-dependent SMAD signaling pathway (hereinafter referred as SMAD pathway) and the non-SMAD signaling pathway (hereinafter referred as non-SMAD pathway) involving direct interactions. In some embodiments relating to wound repair and healing, TGF-β mainly employs the MAPK branch of the non-SMAD pathway to promote epithelial-mesenchymal transition and cell migration, thus facilitating wound repair and healing.

As used herein, the term “SMAD (Small Mothers Against Decapentaplegic)” refers to transcription factors involved in the TGF-β signaling pathway, responsible for regulating cellular proliferation, differentiation, extracellular matrix synthesis, and immune responses. Members of SMAD include SMAD2 and SMAD3, each playing distinct roles in wound healing processes. SMAD2 primarily contributes to anti-fibrotic and anti-inflammatory responses, aiding appropriate wound repair, whereas SMAD3 participates in processes that promote fibrotic tissue growth and scar formation. Once activated, SMAD2/3 translocate into the nucleus, modulating the expression of various genes. Phosphorylated SMAD2/3 enters the cell nucleus to increase the expression of pro-collage, thereby enhancing cell proliferation and migration around the wound, and play an important role in regulating the synthesis and deposition of extracellular matrix.

As used herein, the term “Masson's Trichrome stain” refers to a histological staining technique utilized to distinguish collagen fibers from muscle fibers within tissues. The Masson's Trichrome method employs a combination of acidic and basic dyes, producing distinct coloration patterns in tissue sections. Collagen fibers and cartilage appear blue or green; muscle fibers, keratin, and erythrocytes appear red; and cell nuclei are stained dark brown or black. These color differentiations aid in distinguishing various tissue structures and components.

As used herein, the term “nanozyme” refers to nanoparticles exhibiting enzyme-like catalytic properties. Nanozymes represent a novel category of enzyme-mimicking nanomaterials, characterized by enhanced stability and improved controllability of catalytic activities compared to natural enzymes.

In some embodiments, the present disclosure provides a method of treating skin, which comprises administering to a subject in need thereof a composition, wherein the composition comprises a polymer dot.

In at least one embodiment of the present disclosure, the method further comprises topically administering to the subject a picosecond laser. In some embodiments, the composition and the picosecond laser are topically administering to the subject to enhance wound healing and/or tissue regeneration of the subject.

In at least one embodiment of the present disclosure, the composition is configured to promote skin repair and/or skin regeneration of the subject. In another embodiment of the present disclosure, the composition is configured to enhance wound healing and/or inhibit scar formation on the skin of the subject.

In at least one embodiment of the present disclosure, the polymer dot comprises a carbon dot. In at least one embodiment of the present disclosure, the carbon dot is a dendritic carbon dot.

In at least one embodiment of the present disclosure, the dendritic carbon dot comprises a non-conjugated structure.

In at least one embodiment of the present disclosure, the dendritic carbon dot comprises a first monomer of Formula (I): A₂B₃ (I), wherein A₂ is a dianhydride and B₃ is a polyetheramine.

In at least one embodiment of the present disclosure, the dianhydride comprises a bridged bicyclic olefinic structure or a non-olefinic structure.

In at least one embodiment of the present disclosure, a terminal group of the dendritic carbon dot comprises an amino group, and the first monomer is aliphatic.

In at least one embodiment of the present disclosure, the dendritic carbon dot comprises a second monomer of Formula (II): AB₂ (II), wherein A is an amino group and B₂ is a carboxylic acid, or A is a carboxylic acid and B₂ is an amino group.

In at least one embodiment of the present disclosure, an interior of the dendritic carbon dot comprises an olefinic structure. In at least one embodiment of the present disclosure, the dendritic dot comprises an amino group. In at least one embodiment of the present disclosure, a terminal of the dendritic carbon dot comprises a carboxyl group structure. In at least one embodiment of the present disclosure, the second monomer is aliphatic.

In at least one embodiment of the present disclosure, the composition further comprises a humectant. In some embodiments, the humectant may contain polyethylene glycol with molecular weight of 1000 g/mol to 2000 g/mol, e.g., 1000 g/mol, 1100 g/mol, 1200 g/mol, 1300 g/mol, 1400 g/mol, 1500 g/mol, 1600 g/mol, 1700 g/mol, 1800 g/mol, 1900 g/mol, or 2000 g/mol, but the present disclosure is not limited thereto.

In at least one embodiment of the present disclosure, the composition may further include an auxiliary media configured for irradiation by the picosecond laser. In some embodiments, the auxiliary media may be silica-coated dye particles, gold nanoparticles (AuNPs) in solution or gel, chromophore-loaded hydrogel, liposome-based delivery medium, titanium dioxide or zinc oxide nanoparticle gel, carbon suspension (carbon-based lotion or gel), or black phosphorus or graphene quantum dot formulation, but the present disclosure is not limited thereto.

In certain embodiments, to address problems associated with the use of nanoparticles as drug carriers for skin wound repair and healing (e.g., cytotoxicity), the present disclosure provides a dendritic polymeric nano carbon dot designed to enhance wound repair and healing. The dendritic polymeric nano carbon dot of the present disclosure is synthesized through innovative molecular designs resulting in atypical fluorescent dendritic polymeric carbon dots, including the synthesis of dendritic polymeric carbon dots utilizing all-aliphatic monomers structured as A₂+B₃ or A+B₂. The dendritic polymeric nano carbon dot of the present disclosure exhibits the following advantages: (1) a manufacturing process with high synthesis yields suitable for mass production; (2) small nanoparticle size, allowing easy dispersion in water; (3) atypical fluorescent quantum behavior, making them suitable candidate materials for scavenging free radicals; and (4) internal bond having all-aliphatic and protein-like structures with biological activity.

In certain embodiments, the synthetic yield of the dendritic polymeric nano carbon dot described in the present disclosure ranges from about 5% to about 95%, such as from about 20% to about 90%, from about 30% to about 80%, or from about 40% to about 70%. Specific examples include about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%. However, the present disclosure is not limited thereto.

In certain embodiments, the particle size of the dendritic polymeric nano carbon dot described in the present disclosure ranges from about 1.5 nm to about 15 nm, such as from about 2 nm to about 10 nm, or from about 2.5 nm to about 6.5 nm. Specific examples include about 1.5 nm, about 2.0 nm, about 2.5 nm, about 3.0 nm, about 3.5 nm, about 4.0 nm, about 4.5 nm, about 5.0 nm, about 5.5 nm, about 6.0 nm, about 6.5 nm, about 7.0 nm, about 7.5 nm, about 8.0 nm, about 8.5 nm, about 9.0 nm, about 9.5 nm, about 10.0 nm, about 10.5 nm, about 11.0 nm, about 11.5 nm, about 12.0 nm, about 12.5 nm, about 13.0 nm, about 13.5 nm, about 14.0 nm, about 14.5 nm, or about 15.0 nm. However, the present disclosure is not limited thereto.

An effective drug delivery system ensures that a therapeutic agent enters the body and reaches the targeted site. Topical drug delivery (hereinafter referred as TDD) is a popular administration route due to its convenience and affordability. This method introduces a therapeutic agent onto body surfaces using formulations that can be absorbed by the skin, such as transdermal patches, topical creams, or mucosal sprays. This approach enables easier dosing control and avoids first-pass metabolism effects in the gastrointestinal tract and liver, thereby enhancing the bioavailability of the therapeutic agent. To achieve effective drug transport through the skin, chemical and physical barriers presented by the skin, including the stratum corneum, must be overcome. The present disclosure provides a drug delivery system combining chemical and physical methodologies to optimize wound treatment effects via topical drug administration.

In certain embodiments, the polymeric carbon dot disclosed herein functions as bioactive nanozymes and serves as materials for skin regeneration and healing.

In certain embodiments, the dendritic polymeric nano carbon dot of the present disclosure is dispersed in an aqueous solution, specifically dissolved in phosphate-buffered saline (hereinafter referred as PBS), and formulated as a gel using polyethylene glycol with an average molecular weight of 1000 (hereinafter referred as PEG 1000) at a concentration of 5 mg/mL to produce a viscous liquid. This gel formulation can be applied topically onto the skin. In some embodiments, PEG 1000 is prepared in PBS at a concentration of 5 mg/mL. Additionally, the dendritic polymeric nano carbon dot dressing disclosed herein may also be combined with other physical or chemical treatments as further detailed below.

In at least one embodiment, external physical stimuli such as ultrasound or thermal energy can be employed to enhance permeability of nanoparticle through the skin. For example, laser therapies such as fractional carbon dioxide (CO₂) lasers have demonstrated effectiveness in enhancing drug delivery by ablating superficial skin layers to create microchannels for increased drug penetration. However, fractional CO₂ lasers typically induce significant inflammatory reactions and cause damage to the skin surface, limiting their utility in wound healing applications. In contrast, picosecond lasers not only facilitate drug delivery but also possess wound healing properties. Picosecond lasers produce rapid photothermal effects with minimal thermal damage and do not create open wounds. Following application at various wavelengths, picosecond lasers induce laser-induced optical breakdown (hereinafter referred as LIOB) in the skin, triggering repair mechanisms, collagen remodeling, and neocollagenesis without promoting fibrosis. Moreover, picosecond lasers activate the TGF-β/SMAD signaling pathway, enhancing nuclear collagen expression and suppressing inflammation-related transcription factors, thereby promoting wound healing. The clinical effects differ according to the laser wavelength: picosecond lasers with a wavelength of 532 nm targets superficial skin layers; and a wavelength of 1,064 nm targets on the deeper superficial dermis but is easily absorbed by hemoglobin, often causing visible erythema and petechiae with prolonged recovery times; and a wavelength of 755 nm acts on both epidermal and dermal layers, causing minimal surface damage without visible petechiae. Therefore, the present disclosure utilizes a picosecond laser combined with a microlens array as a pretreatment strategy to enhance the penetration of the dendritic polymeric nano carbon dot of the present disclosure into the skin. In addition to therapeutic benefits, picosecond lasers serve as physical enhancers for topical drug delivery.

In some embodiments, the 755 nm picosecond laser may be combined with a diffractive lens array.

In some embodiments, the picosecond laser disclosed herein is applied in 100 to 1,000 pulses, such as 400 to 600 pulses, or 200 to 800 pulses. Exemplary pulse counts include about 100, 200, 300, 400, 500, 600, 700, 800, 900, or 1,000 pulses, though the present disclosure is not limited thereto.

In some embodiments, the picosecond laser output energy ranges from about 0.25 J/cm² to about 1.50 J/cm², preferably from about 0.50 J/cm² to about 0.80 J/cm² or from about 0.40 J/cm² to about 0.90 J/cm². Exemplary energy values include about 0.25 J/cm², 0.30 J/cm², 0.35 J/cm², 0.40 J/cm², 0.45 J/cm², 0.50 J/cm², 0.55 J/cm², 0.60 J/cm², 0.65 J/cm², 0.70 J/cm², 0.75 J/cm², 0.80 J/cm², 0.85 J/cm², 0.90 J/cm², 0.95 J/cm², 1.00 J/cm², 1.05 J/cm², 1.10 J/cm², 1.15 J/cm², 1.20 J/cm², 1.25 J/cm², 1.30 J/cm², 1.35 J/cm², 1.40J/cm², 1.45 J/cm², or 1.50 J/cm². However, the present disclosure is not limited thereto.

In some embodiments, the dendritic carbon dot has a concentration of 1 mg/ml to 10 mg/ml. Exemplary concentrations include about 1 mg/ml, 2 mg/ml, 3 mg/ml, 4 mg/ml, 5 mg/ml, 6 mg/ml, 7 mg/ml, 8 mg/ml, 9 mg/ml, or 10 mg/ml. However, the present disclosure is not limited thereto.

In some embodiments, the present disclosure provides a method combining the picosecond laser treatment with the dendritic polymeric nano carbon dot for wound repair or healing, achieving synergistic effects. This combination provides anti-inflammatory activity, regulates collagen production, promotes epidermal and dermal repair, and enhances angiogenesis.

In some embodiments, the optical breakdown induced by the picosecond laser creates reservoirs within the skin for the dendritic polymeric nano carbon dot disclosed herein, thus enhancing penetration and pharmacological efficacy of the dressing. This ensures effective distribution within epidermal and dermal layers to enhance repair of damaged tissue.

In some embodiments, nude mice serve as animal models for evaluating wound healing by pretreatment with picosecond lasers and combined with the dendritic polymeric nano carbon dot disclosed herein. Picosecond laser-induced optical breakdown stimulates collagen proliferation and tissue repair. Due to the ultrashort duration of laser-skin interactions, picosecond lasers produce microscopic cavitation through supersonic shockwaves, improving skin permeability for drug delivery and enhancing penetration of subsequently applied dendritic polymeric nano carbon dot dressings. Furthermore, keratinocytes respond to injury by releasing growth factors and activating epidermal repair pathways, thus stimulating and modulating the optical breakdown response. This allows the dendritic polymeric nano carbon dot dressing disclosed herein to be effectively distributed into laser-induced vacuoles within epidermal and dermal layers. Therefore, pretreatment with picosecond lasers, and combined with the dendritic polymeric nano carbon dot disclosed herein, results in synergistic therapeutic efficacy.

### EXAMPLES

The following examples illustrate specific embodiments of the present disclosure in further detail but should not be construed as limiting the scope thereof.

### Materials and Methods

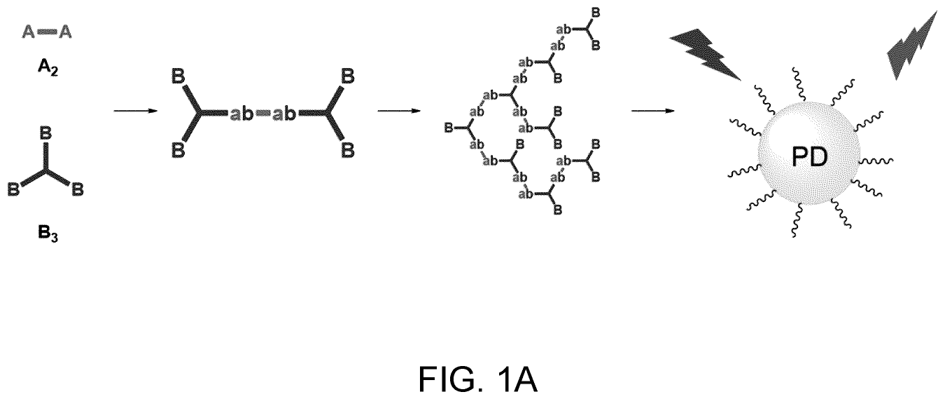

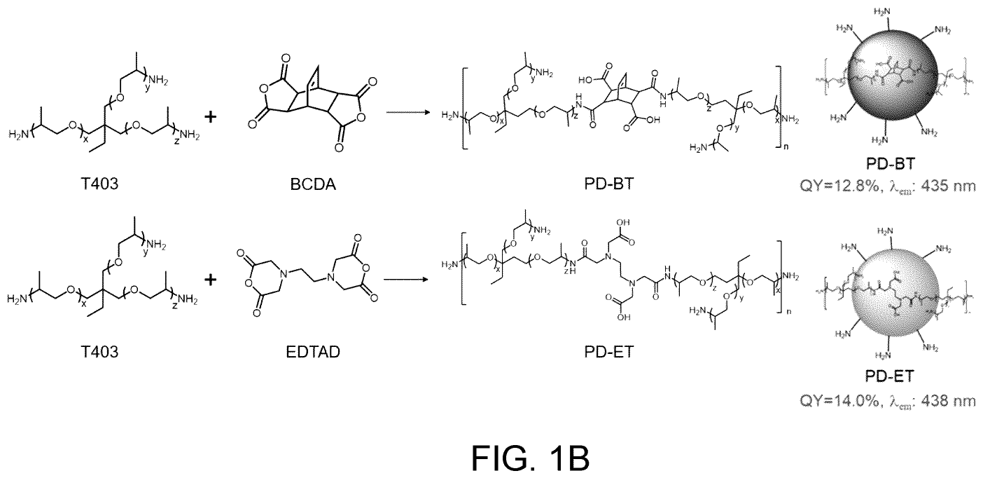

Two dendritic polymeric carbon dots were synthesized from all-aliphatic monomers having an A₂+B₃ structures (shown as FIG. 1A). These polymer dots exhibited non-conjugated hyperbranched aliphatic and alicyclic structures. Two types of dianhydrides as A₂ components were utilized: a bridged bicyclic alkene (e.g., bicyclo[2.2.2]oct-7-ene-2,3,5,6-tetracarboxylic dianhydride, hereinafter referred as BCDA) and a non-alkene (e.g., ethylenediaminetetraacetic dianhydride, hereinafter referred as EDTAD). Polyetheramine (Jeffamine T403, hereinafter referred as T403 and shown as FIG. 1B) served as the B₃ component. These polymer dots have non-conjugated hyperbranched poly(amido acid) (or poly(amino acid)) structures with terminal amino groups and are referred to herein as PD-BT (bridged bicyclic alkene serves as A₂ component; upper portion of FIG. 1B) and PD-ET (non-alkene serves as A₂ component; lower portion of FIG. 1B).

PD-BT and PD-ET demonstrated atypical fluorescence. PD-ET fluorescence intensity varied with pH, whereas PD-BT, due to its rigid aliphatic bridged bicyclic structure, was less sensitive to pH changes. The net charge of PD-BT and PD-ET was adjustable by altering pH. The quantum yields (hereinafter referred as QY) at emission wavelengths (hereinafter referred as em) of 435 nm (PD-BT) and 438 nm (PD-ET) were 12.8% and 14.0%, respectively (shown as FIG. 1B). Transmission electron microscopy (hereinafter referred as TEM) indicated particle sizes of 3-5 nm for PD-BT and PD-ET. PD-BT (50 μg/mL) and PD-ET (500 μg/mL) exhibited no cytotoxicity in human breast cancer cells (MCF-7) and human keratinocytes (HaCaT). Confocal microscopic imaging showed that PD-BT or PD-ET could be observed inside cells 6 hours after administration.

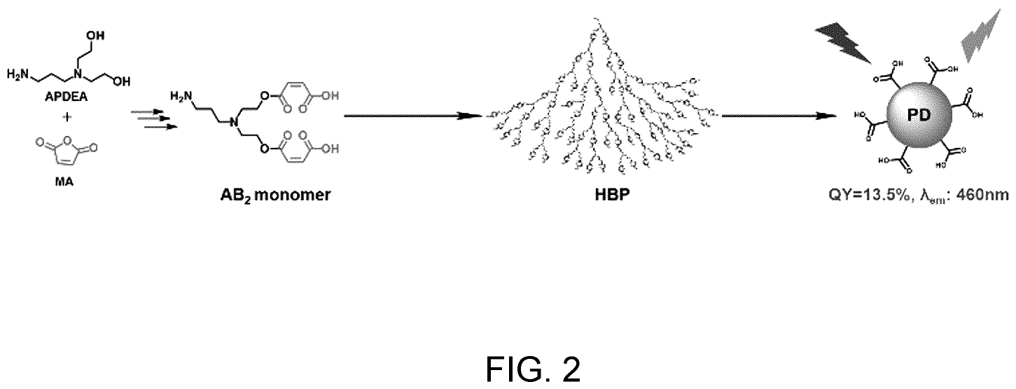

AB₂ monomer composed of N-(3-aminopropyl)diethanolamine (hereinafter referred as APDEA) and maleic anhydride (hereinafter referred as MA) was self-polymerized into a hyperbranched polymer (hereinafter referred as HBP), subsequently self-assembled into dendritic polymeric carbon dots. These dots exhibited emission at 460 nm, a quantum yield of 13.5%, and particle sizes around 9.1 nm (shown as FIG. 2).

Antioxidant activity of HBP was observed at concentrations of 0.6 mg/mL and 1.5 mg/mL in assays for hydroxyl radical, peroxide, and superoxide anion scavenging activities.

Ideal topical drugs require effective skin penetration and stable biodistribution. Particle size of the nanoparticle critically affects topical drug delivery (hereinafter referred as TDD). Skin penetration occurs through intracellular/transcellular, intercellular, and appendageal routes. Nanoparticles smaller than 4 nm bypass charge-related limitations, penetrating the stratum corneum (hereinafter referred as SC) and reaching the dermis.

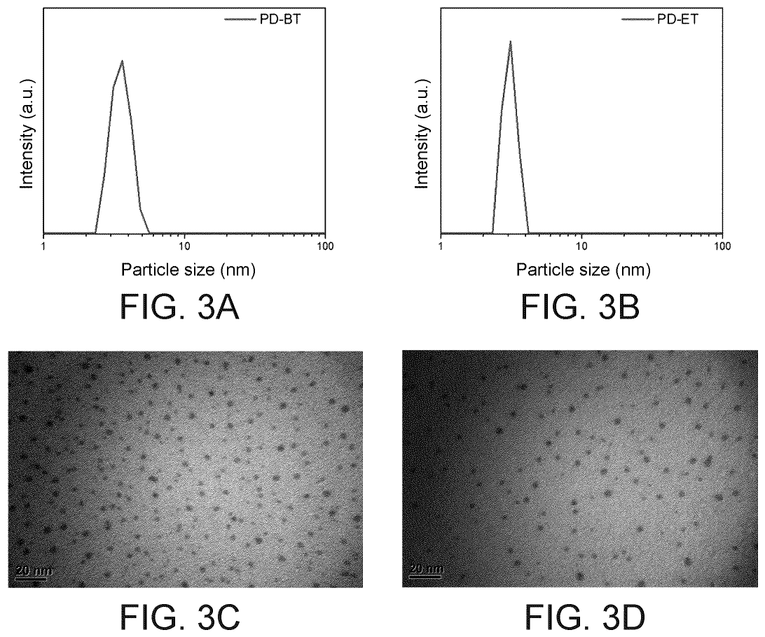

Dynamic light scattering (hereinafter referred as DLS) analysis showed particle sizes of approximately 3 nm for PD-BT and PD-ET (shown as FIG. 3A and FIG. 3B; a.u.: arbitrary unit). High-resolution transmission electron microscopy (hereinafter referred as HRTEM) revealed morphologies of PD-BT (shown as FIG. 3C) and PD-ET (FIG. 3D).

Franz diffusion cell experiments showed HBP skin permeation in mice increased over time, reaching a maximum (0.12 mg/cm²) at 30 hours, indicating effective skin permeability.

The dendritic polymeric carbon dot disclosed herein offers ease of synthesis, excellent water solubility, high biocompatibility, efficient skin penetration, metabolic stability, stable photoluminescence in vitro and in vivo, atypical fluorescence properties, and modifiable surface functional groups. These characteristics render them suitable for bioimaging, fluorescent-labeled drug carrier, and nanomedicine applications.

In certain embodiments, the composition disclosed herein additionally comprises thickening agents or humectants such as polyethylene glycol 1000 (hereinafter referred as PEG 1000), acrylic acid-based polymers (Carbopol®), sodium hyaluronate, or sodium alginate.

PEG 1000 serves as a thickening agent or humectant for the dendritic polymer carbon dot disclosed herein, and maintaining wound moisture during the dendritic polymeric carbon dot application, facilitating cell migration, enhancing wound repair, and reducing the formation of scar and crack. PEG 1000 is a stable, water-soluble polymer used widely as a solvent, thickening agent, plasticizer, lubricant, and dispersant.

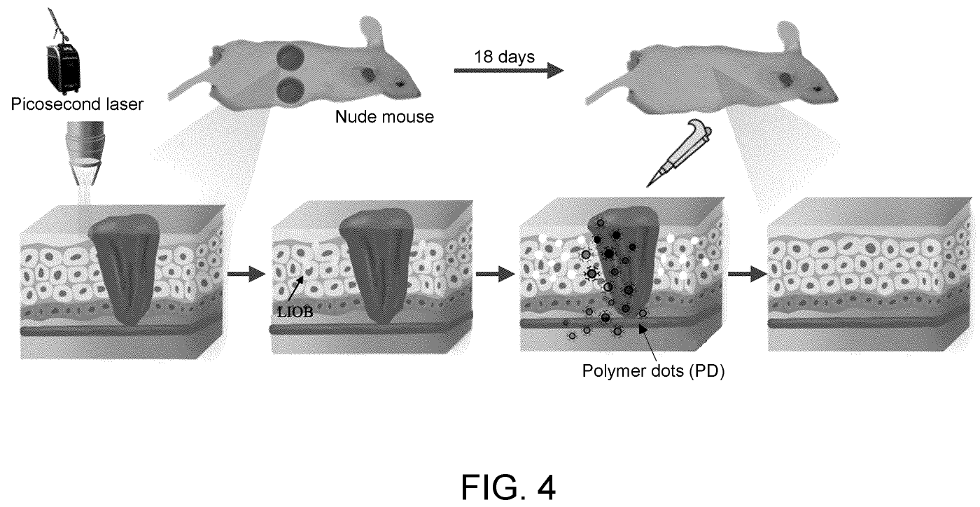

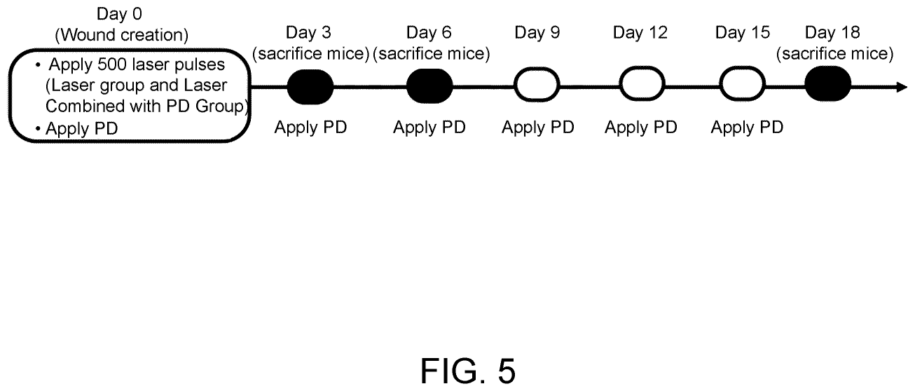

Female athymic nude mice (BALB/cAnN.Cg-Foxn1nu/CrlNarl; strain ID: RMRC12005), purchased from the National Laboratory Animal Center (hereinafter referred as NLAC), were used as a model organism to evaluate the efficacy and activity of the disclosed dendritic polymer dot. After sterile preparation of the surgical site on the dorsal skin, the mice were randomly divided into five groups: control group, PEG 1000 group, PD group, laser group, and laser combined with PD group. The experimental procedures for each group are described in detail below and illustrated in FIGS. 4 and 5.

After anesthetizing the nude mice in the control group using isoflurane, two full-thickness skin wounds were created on the backs of the mice using a 6 mm biopsy punch. To inhibit wound contraction, a 0.45 mm silicone ring (inner diameter: 10 mm; outer diameter: 18 mm) was used to fix the wounds. The silicone ring was fixed in place using a non-adhesive silicone patch and sutured with six stitches of 6-0 nylon. Finally, the wounds were covered with a Tegaderm waterproof and breathable dressing (3M Health Care, MN, USA).

After anesthetizing the nude mice in the PEG 1000 group using isoflurane, two full-thickness skin wounds were created on the backs of the mice using a 6 mm biopsy punch. To inhibit wound contraction, a 0.45 mm silicone ring (inner diameter: 10 mm; outer diameter: 18 mm) was used to fix the wounds. The silicone ring was fixed in place using a non-adhesive silicone patch and sutured with six stitches of 6-0 nylon. PEG 1000 (application volume: 200 μL) was applied to the entire dorsal skin of the nude mice in the PEG 1000 group, and re-applied PEG 1000 every three days (i.e., on days 0, 3, 6, 9, 12, and 15). Finally, the wounds were covered with a Tegaderm waterproof and breathable dressing (3M Health Care, MN, USA).

After anesthetizing the nude mice in the PD group using isoflurane, two full-thickness skin wounds were created on the backs of the mice using a 6 mm biopsy punch. To inhibit wound contraction, a 0.45 mm silicone ring (inner diameter: 10 mm; outer diameter: 18 mm) was used to fix the wounds. The silicone ring was fixed in place using a non-adhesive silicone patch and sutured with six stitches of 6-0 nylon. The disclosed polymer carbon dots (concentration: 5 mg/mL; application volume: 200 μL) were applied to the entire dorsal skin of the nude mice in the PD group and re-applied the disclosed polymer carbon dots every three days (i.e., on days 0, 3, 6, 9, 12, and 15). Finally, the wounds were covered with a Tegaderm waterproof and breathable dressing (3M Health Care, MN, USA).

After anesthetizing the nude mice in the laser group using isoflurane, two full-thickness skin wounds were created on the backs of the mice using a 6 mm biopsy punch. A total of 500 pulses of picosecond laser were uniformly applied to the dorsal skin of the nude mice in the laser group (wavelength: 755 nm; spot size: 6 mm; output energy: 0.71 J/cm²). To inhibit wound contraction, a 0.45 mm silicone ring (inner diameter: 10 mm; outer diameter: 18 mm) was used to fix the wounds. The silicone ring was fixed in place using a non-adhesive silicone patch and sutured with six stitches of 6-0 nylon. Finally, the wounds were covered with a Tegaderm waterproof and breathable dressing (3M Health Care, MN, USA).

In some embodiments, after anesthetizing the nude mice in the laser combined with PD group using isoflurane, two full-thickness skin wounds were created on the backs of the mice using a 6 mm biopsy punch. A total of 500 pulses of picosecond laser were uniformly applied to the dorsal skin of the nude mice in the laser combined with PD group (wavelength: 755 nm; spot size: 6 mm; output energy: 0.71 J/cm²). To inhibit wound contraction, a 0.45 mm silicone ring (inner diameter: 10 mm; outer diameter: 18 mm) was used to fix the wounds. The silicone ring was fixed in place using a non-adhesive silicone patch and sutured with six stitches of 6-0 nylon. The disclosed polymer carbon dots (concentration: 5 mg/mL; application volume: 200 μL) were then applied to the entire dorsal skin of the nude mice in the laser combined with PD group and re-applied the disclosed polymer carbon dots every three days (i.e., on days 0, 3, 6, 9, 12, and 15). Finally, the wounds were covered with a Tegaderm waterproof and breathable dressing (3M Health Care, MN, USA).

As shown in FIG. 5, on days 3, 6, and 18 after wound creation, the nude mice in each group were euthanized following anesthesia with carbon dioxide. The wound sites and wound edges were excised for analysis. Nude mice that were not euthanized continued to receive treatment with the disclosed polymer carbon dots.

In the present disclosure, the excised wound sites and wound edges from each group of nude mice were subjected to quantitative analysis of target proteins using enzyme-linked immunosorbent assay (hereinafter referred as ELISA). Specifically, the harvested tissues were placed into grinding tubes and added in Dulbecco's phosphate-buffered saline (hereinafter referred as DPBS) containing protease inhibitors, and then were grinded and homogenized. The homogenates were then centrifuged at 16,000×g at 4° C. for 20 minutes. The resulting supernatants were transferred to a new microcentrifuge tubes and centrifuged again under the same conditions. The final supernatants were collected and subjected to protein quantification using a Bio-Rad Protein Assay Dye Reagent (#5000006), and the resulting samples were stored at −80° C.

In the present disclosure, the excised wound tissues and wound edges from each group of nude mice were subjected to immunohistochemical staining. The tissue samples were first fixed in 10% formalin, embedded in paraffin, and stained for MMP-9, CD31, fibronectin, phospho-p38 MAPK, and CD34. The staining procedures and reagents used are described in Tables 1, 2, 3, 4, and 5, respectively.

TABLE 1

| | | Reagent Name | Reaction time |
|---|---|---|---|
| Reagent | Reagent Kit | TAHC04D | |
| | Primary Antibody | Anti-MMP-9 antibody | |
| | | (Abcam, ab38898) | |
| | | (Dilution factor: 200X) | |
| | H₂O₂ | | 10 minutes |
| Pretreatment | Tris EDTA (pH 9.0) | | 12 minutes |
| Staining | Blocking | | 60 minutes |
| Procedure | Primary Antibody Incubation | | 16 hours (4° C.) |
| | Secondary Antibody Incubation | | 30 minutes |
| | Chromogen Development | 3,3′-Diaminobenzidine | 30 seconds |
| | | (hereinafter referred as DAB) | |
| | Counterstaining | Hematoxylin | 5 seconds |

TABLE 2

| | | Reagent Name | Reaction time |
|---|---|---|---|
| Reagent | Reagent Kit | TAHC04D | |
| | Primary Antibody | Anti-CD31 antibody | |
| | | (Thermo, PA5-32321) | |
| | | (Dilution factor: 100X) | |
| | H₂O₂ | | 10 minutes |
| Pretreatment | Citrate buffer (pH 6.0) | | 12 minutes |
| Staining | Blocking | | 60 minutes |
| Procedure | Primary Antibody | | 16 hours |
| | Incubation | | (room temp) |
| | Secondary Antibody | | 30 minutes |
| | Incubation | | |
| | Chromogen Treatment | 3,3′-Diaminobenzidine | 10 minutes |
| | | (hereinafter referred as | |
| | | DAB) | |
| | Counterstaining | Hematoxylin | 5 seconds |

TABLE 3

| | | Reagent Name | Reaction time |
|---|---|---|---|
| Reagent | Reagent Kit | TAHC04D | |
| | Primary Antibody | Anti-fibronectin antibody | |
| | | (GeneTex, GTX112794) | |
| | | (Dilution factor: 500X) | |
| | H₂O₂ | | 10 minutes |
| Pretreatment | Citrate buffer (pH 6.0) | | 12 minutes |
| Staining Procedure | Blocking | | 60 minutes |
| | Primary Antibody Incubation | | 16 hours (4° C.) |
| | Secondary Antibody Incubation | | 30 minutes |
| | Chromogen Development | 3,3′-Diaminobenzidine | 2 minutes |
| | | (hereinafter referred as DAB) | |
| | Counterstaining | Hematoxylin | 5 seconds |

TABLE 4

| | | Reagent Name | Reaction time |
|---|---|---|---|
| Reagent | Reagent kit | TAHC04D | |
| | Primary Antibody | Anti-phospho-p38 MAPK | |
| | | antibody (Thermo Fisher, | |
| | | #BS-2210R), dilution | |
| | | factor: 200 (200X) | |
| | H₂O₂ | | 10minutes |
| Pretreatment | Citrate buffer (pH 6.0) | | 12 minutes |
| Staining Procedure | Blocking | | 60 minutes |
| | Primary Antibody | | 16 hours (4° C.) |
| | Incubation | | |
| | Secondary Antibody Incubation | | 30 minutes |
| | Chromogen Development | 3,3′-Diaminobenzidine | 10 minutes |
| | | (hereinafter referred as DAB) | |
| | Counterstaining | hematoxylin | 5 seconds |

TABLE 5

| | | Reagent Name | Reaction Time |
|---|---|---|---|
| Reagent | Reagent kit | TAHC04D | |
| | Primary Antibody | Anti-CD34 antibody | |
| | | (Thermo Fisher, | |
| | | #PA5-85917) (Dilution | |
| | | factor: 3000X) | |
| | H₂O₂ | | 10 minutes |
| Pretreatment | Citrate Buffer (pH 6.0) | | 12 minutes |
| Staining Procedure | Blocking | | 60 minutes |
| | Primary Antibody Incubation | | 1 hour (room |
| | | | temperature) |
| | Secondary Antibody Incubation | | 30 minutes |
| | Chromogen Development | 3,3′-Diaminobenzidine | 3 minutes |
| | | (hereinafter referred as DAB) | |
| | Counterstaining | Hematoxylin | 5 seconds |

In the present disclosure, the harvested wound and wound edge tissues from the aforementioned groups of nude mice were subjected to Masson's Trichrome staining. First, the paraffin-embedded tissue samples were sequentially deparaffinized and rehydrated using 100% ethanol, 95% ethanol, and 70% ethanol, followed by washing with distilled water and rinsing with tap water for 5 to 10 minutes. The tissue samples were then stained in Weigert iron hematoxylin dye solution for 10 minutes, rinsed with tap water for 10 minutes, and subsequently washed with distilled water. The washed samples were stained in Biebrich Scarlet stain solution for 10 to 15 minutes, followed by brief rinsing with distilled water to remove excess stain. The washed tissue samples were then differentiated in phosphomolybdic-phosphotungstic acid solution for 3 to 5 minutes until collagen appeared clear (which could be verified under a microscope). The tissue samples were transferred to aniline blue solution for 5 minutes of staining, briefly rinsed with distilled water, and differentiated in 1% acetic acid solution for 2 minutes. Finally, the tissue samples were dehydrated using 95% ethanol and absolute ethanol, and then placed in xylene until transparent, and mounted using a resin-based mounting medium. Through Masson's Trichrome staining, the structural components of the tissue samples were stained, wherein collagen appeared blue, nuclei appeared black, and muscle fibers, cytoplasm, and keratin appeared red.

Statistical analysis of the experimental results on the use of the polymer carbon dots and/or laser described in the present disclosure was conducted using GraphPad Prism 10. One-way ANOVA and two-way ANOVA were used to compare the groups (i.e., control group, PEG 1000 group, PD group, laser group, and laser combined with PD group). Tukey's Honest Significant Difference (hereinafter referred as HSD) test was used for multiple comparisons and automatic correction. A P-value of less than 0.05 was considered statistically significant among the groups.

The 755 nm picosecond laser used in the present disclosure is combined with a diffractive lens array (hereinafter referred as DLA) (PicoSure®, Cynosure, Westford, Massachusetts, USA).

The present disclosure utilizes optical coherence tomography (hereinafter referred as OCT) to automatically identify wounds within tissue layers and provide relevant wound information, including wound location, size, and morphology. Based on the three-dimensional images obtained by scanning, various skin parameters can be quantitatively evaluated, including the thickness of the stratum corneum and epidermis, collagen content, vascular density, pore/hair follicle size, and skin roughness. Additionally, the volume of the wound bed can be calculated using the known wound width and depth. Thus, observing the skin layers of a living subject through OCT enables real-time imaging and wound status measurement without sacrificing the animal.

### Analysis and Results

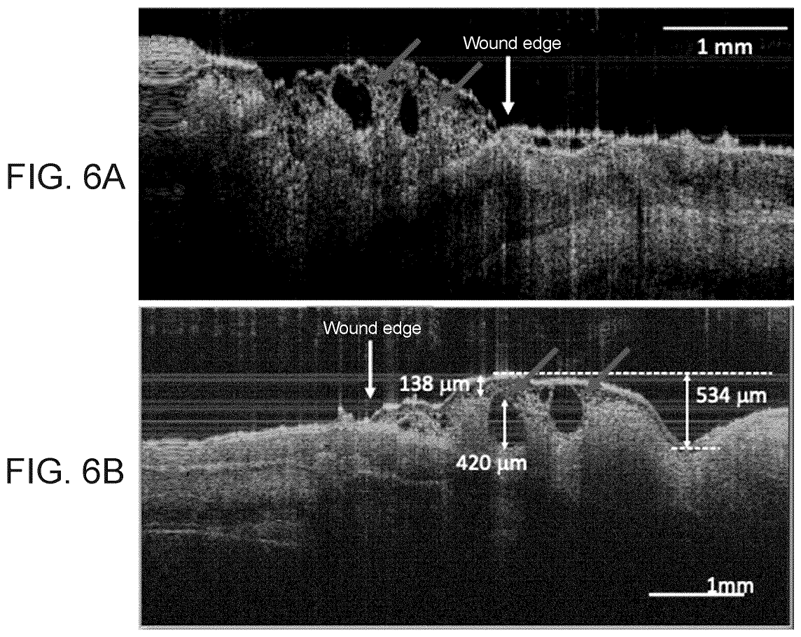

In some embodiments, the wound created on the skin of a living nude mouse on day 0 was observed using optical coherence tomography (OCT) (OPXION Technology Inc., Taiwan). As shown in FIG. 6A (with the wound edge indicated by white arrows), vacuoles appeared at the junction of the epidermis and dermis after picosecond laser treatment. These vacuoles are the result of laser-induced optical breakdown (as indicated by red arrows). FIG. 6B (with the wound edge indicated by white arrows) shows that the vacuoles by laser-induced optical breakdown remained visible on day 3 post-laser treatment, and the skin structure remained intact without severe damage and well-arranged.

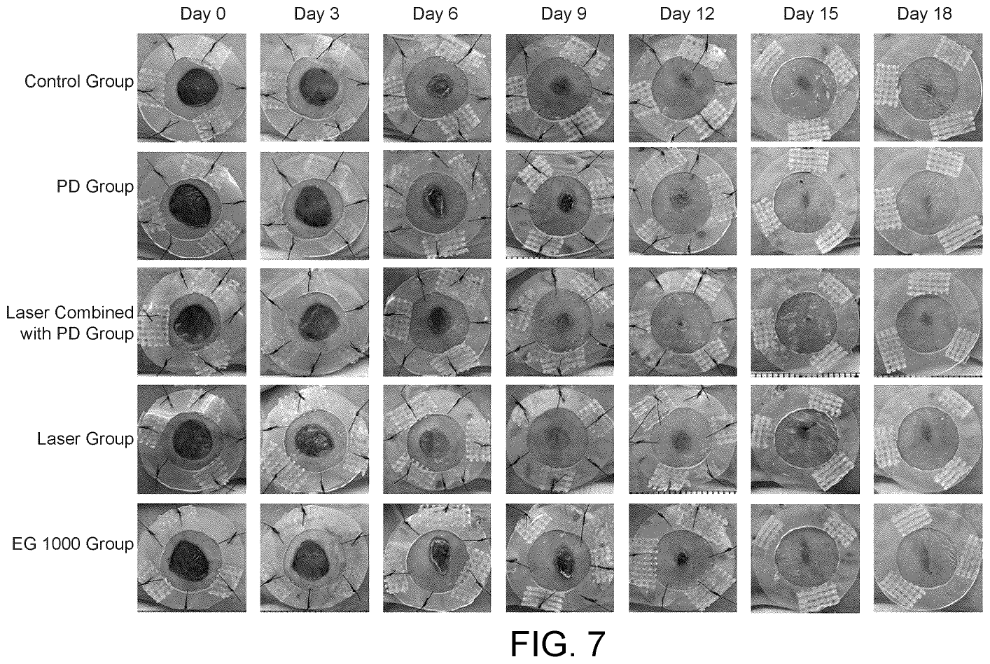

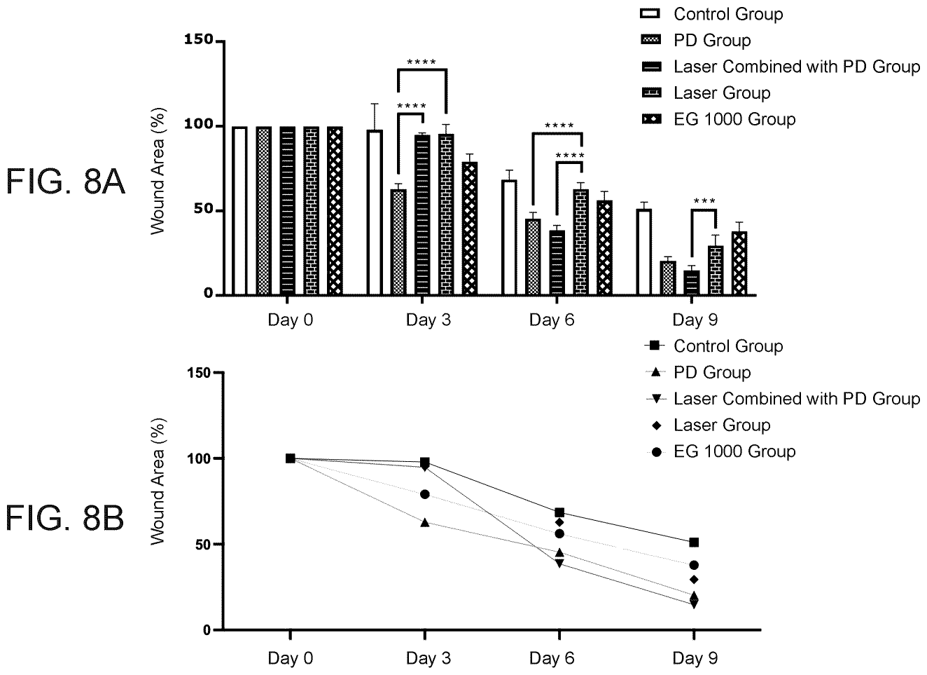

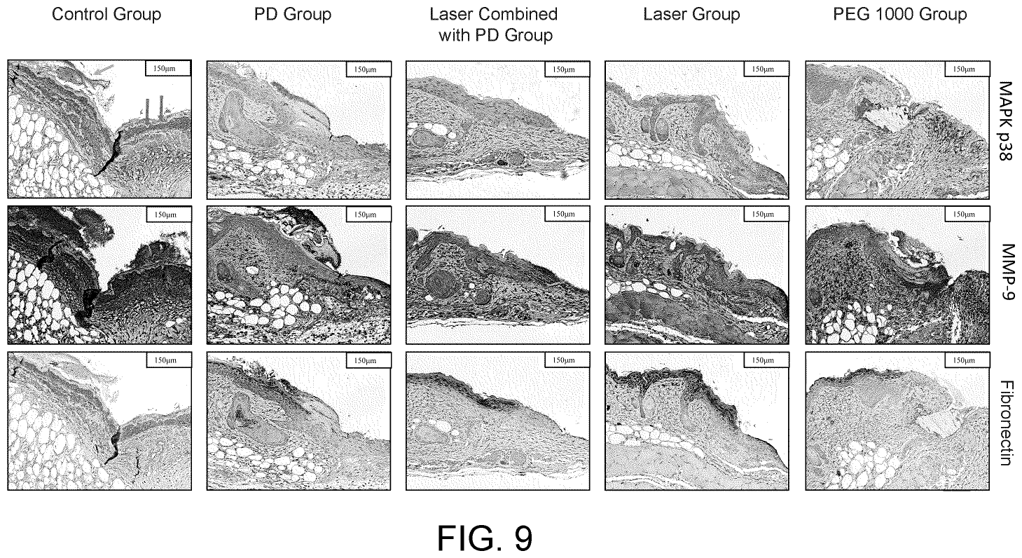

The wound healing and repair process on the skin of nude mice was analyzed using ImageJ, as shown in FIG. 7 and FIGS. 8A and 8B. Simultaneous application of PD and picosecond laser to the skin (laser combined with PD group) promoted wound healing and repair. The data of day 3 shows the wound area of the control group remained similar to that of day 0 (approximately 98%), while the wound areas of other groups were about 75-90%, likely due to the untreated wounds being in the inflammatory phase, resulting in larger wound areas. The PD-treated group (PD group) showed the smallest wound area, possibly due to the anti-inflammatory effect of PD shortening the inflammatory phase. Next was the PEG 1000 group, where PEG 1000, as a thickening agent for PD, acted as a moisturizer to keep the wound moist, having a positive effect on wound healing. The laser-treated groups (laser group and laser combined with PD group) had relatively larger wound areas, possibly due to mild damage to the stratum corneum after picosecond laser treatment on early stage.

On day 6, as shown in FIG. 7 and FIGS. 8A and 8B, the laser combined with PD group exhibited a significantly improved wound healing effect compared with other groups, with wound area reduced to approximately 50%. The followed is the PD group. The laser group and PEG 1000 group showed similar effects, possibly because the wounds were in the proliferative phase on day 6, and the optical breakdown induced by the picosecond laser played a regulatory role that assisted in the sustained release of PD. Therefore, both the laser combined with PD group and the laser group demonstrated advantages in wound healing and repair.

As shown in FIG. 7 and FIGS. 8A and 8B, by day 18, all wounds in each group had healed. Observation of scar formation in each group revealed that the control group and the PEG 1000 group displayed more prominent scars; in contrast, the laser group and the PD group exhibited fewer scars; and the laser combined with PD group showed the least and most complete scar reduction. This may be due to the application of picosecond laser and PD, both of which regulate the expression level of collagen in the skin, promoting uniform collagen generation and reducing collagen translucency. In summary, the treated groups (i.e., PD group, laser group, and laser combined with PD group) showed reduced inflammation compared with the control group. The combination therapy of picosecond laser and PD (i.e., laser combined with PD group) demonstrated superior wound healing effects compared with the use of picosecond laser (laser group) or PD (PD group) alone. Furthermore, both picosecond laser application and PD treatment facilitated wound repair and form fewer scars after healing, which can minimize scar formation.

The combination therapy of picosecond laser treatment and PD treatment established by the present disclosure (i.e., laser combined with PD group) showed significantly better healing effects in nude mouse skin wound repair compared with other groups (i.e., control group, PD group, laser group, and PEG 1000 group). Therefore, the relevant mechanisms of wound repair and healing can be considered to explore how the combination therapy involving the picosecond laser and the disclosed polymer dots (PD) promotes wound repair and healing. According to previous studies, picosecond laser treatment of the skin facilitates epidermal repair and collagen synthesis through the SMAD signaling pathway. In contrast, treatment with polymer carbon dots promotes epithelial-mesenchymal transition (EMT) through the MAPK p38 in a non-SMAD signaling pathway. Taken together, the combination therapy of picosecond laser and PD disclosed herein exerts a synergistic effect on wound repair and healing primarily through TGF-β of both SMAD and non-SMAD signaling pathways.

To evaluate the wound healing efficacy of each group in the nude mouse wound model disclosed herein, immunohistochemistry staining was performed on the wound sites and surrounding tissues, including staining for MAPK p38, MMP-9, fibronectin, CD31, and CD34. The following paragraphs further describe the relationship between these markers and wound healing and repair.

In some embodiments, MAPK p38 participates in the regulation of inflammation and promotes cell proliferation and migration during the early stages of wound healing. Through its signaling pathway, MAPK p38 regulates the synthesis and secretion of MMP-9 and fibronectin. MMP-9 is a type IV collagenase whose expression increases in chronic wounds and can disrupt basement membrane protein's structure, thereby hindering keratinocyte migration, adhesion, and epidermal reconstruction. As wound healing progresses, MMP-9 expression decreases. Fibronectin is an adhesive molecule that binds to cell surface receptors and plays a role in ECM formation during the initial stage of wound healing. It also mediates the migration and proliferation of epidermal cells and tissue regeneration, supporting wound structure reconstruction and the transition to type I collagen.

As shown in FIG. 9, immunohistochemistry was performed on Day 3 after wound creation in the skin of nude mice. The red arrows indicate inflammatory cells, while the green arrows indicate the site of scabbing. The control group and PEG 1000 group exhibited higher expression levels of MAPK p38 and MMP-9. In the control group, a large number of inflammatory cells and scabs were observed at the wound edges, indicating that the control group and PEG 1000 group remained in the early inflammatory phase. In contrast, the PD group, laser group, and laser combined with PD group exhibited reduced expression levels of MAPK p38 and MMP-9, indicating that by Day 3 post-wounding, these groups had transitioned to the post-inflammatory phase and begun tissue repair. At this stage, MAPK p38 is involved in other biological processes, such as cell proliferation, cell migration, and extracellular matrix synthesis. Fibronectin, a marker for epithelial-mesenchymal transition (EMT), was more highly expressed in the PD group, laser group, and laser combined with PD group compared to the control group and PEG 1000 group, indicating that these treated groups had only mild inflammation and had entered tissue regeneration and matrix remodeling phases by Day 3.

Angiogenesis is a critical process in tissue repair, as newly formed blood vessels supply oxygen and nutrients and support the regeneration of damaged tissue. CD31 and CD34 can be used to evaluate angiogenesis. CD31 is a highly specific marker for endothelial cell differentiation and is primarily expressed in vascular endothelial cells, where it promotes Promote the aggregation of endothelial cells and the formation of vascular structure. CD34 is a highly sensitive marker for endothelial cells, mainly expressed in perivascular mesenchymal cells and fibroblasts, and serves as a marker for cell adhesion, mesenchymal cells, and fibroblasts.

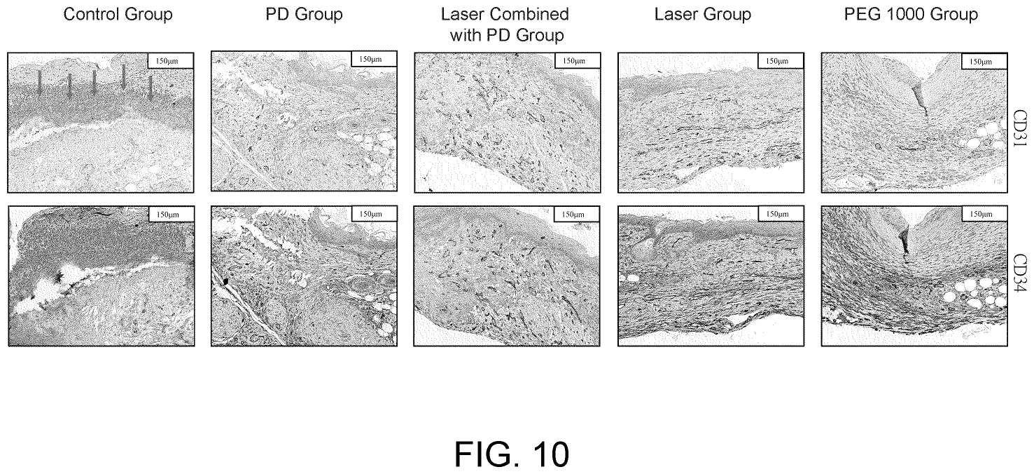

As shown in FIG. 10, immunohistochemistry was performed on Day 6 after wound creation in the skin of nude mice. The red arrows indicate inflammatory cells. The control group and PEG 1000 group showed limited neovascularization in the dermis, indicating that these groups remained in the inflammatory phase, with edema and hemorrhage observed in the surrounding tissue. The PD group, laser group, and laser combined with PD group exhibited significantly increased expression of CD31, with the laser combined with PD group showing the most neovascularization and highest vascular density. These results indicate that during the tissue remodeling phase, treatment with picosecond laser and PD promoted angiogenesis. The control group exhibited only small amount of CD34 expression, while the PEG 1000 group showed higher CD34 expression localized at the base of the dermis. In contrast, the PD group, laser group, and laser combined with PD group exhibited significantly higher CD34 expression. These results suggest that on Day 6 after wound creation, the control group remained in the inflammatory phase, the PEG 1000 group showed signs of excessive fibrosis, and the PD group, laser group, and laser combined with PD groups were in the tissue remodeling phase, undergoing active angiogenesis and tissue regeneration at the wound and surrounding areas.

Collagen plays a role in hemostasis and inflammation regulation. Damage to collagen can stimulate coagulation, leading to fibrin clot formation to stop bleeding. Collagen can exert anti-inflammatory effects and promote angiogenesis through RNA signaling pathways. Type I and type IV collagens can enhance neutrophil activity, phagocytosis, and cytokine secretion, thereby clearing inflammation.

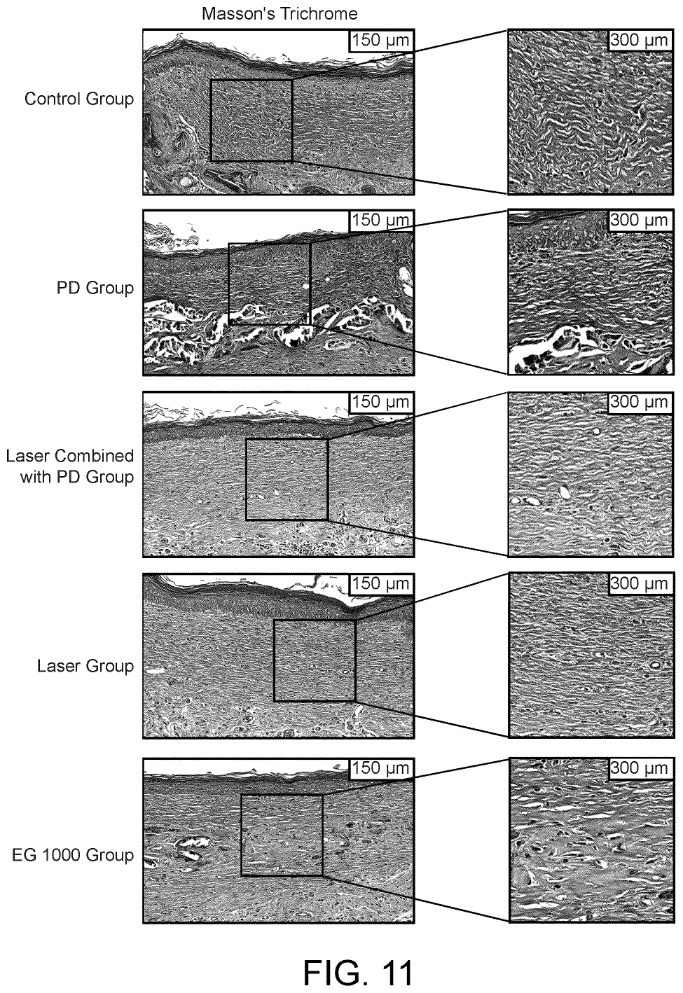

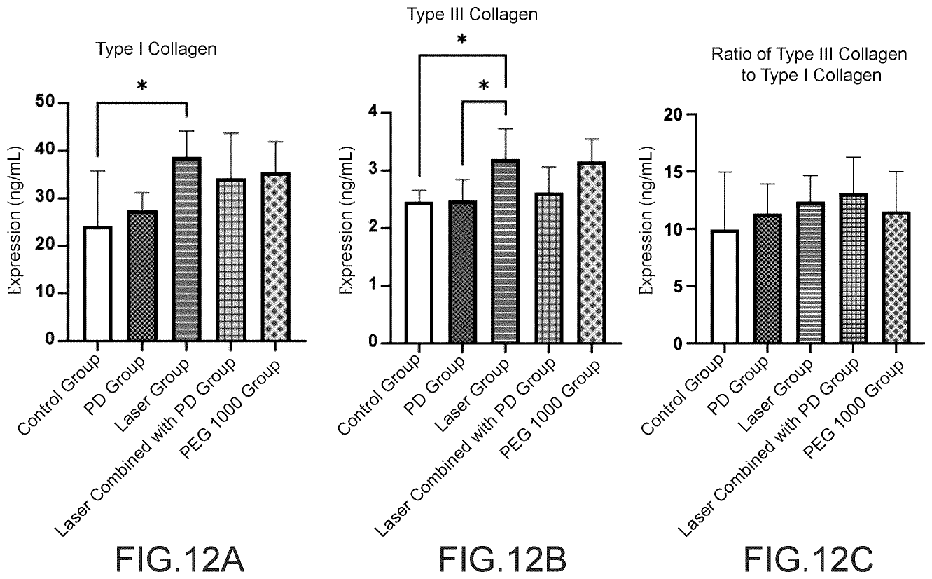

To further validate the aforementioned immunohistochemistry results, Masson's Trichrome staining (shown as FIG. 11) and ELISA quantification (shown as FIG. 12A, FIG. 12B, and FIG. 12C) were conducted on Day 18 after wound creation to evaluate collagen expression in the wound area. FIG. 11 shows the Masson's Trichrome staining results on Day 18 after wound creation in the skin of nude mice. Collagen appears blue, nuclei appear black, and muscle fibers, cytoplasm, and keratin appear red. The control group and PEG 1000 group exhibited a large amount of disorganized, broad, and loosely structured collagen that was inconsistent with the orientation of the wound cross-section, leading to scar formation. In contrast, the PD group, laser group, and laser combined with PD group displayed denser and more orderly aligned collagen fibers. In normal tissue, collagens are arranged in an organized manner, whereas scar formation is primarily due to newly formed collagens aligning in directions inconsistent with the original tissue. Scar formation is influenced by multiple factors, including the extent of fibrotic tissue proliferation, changes in tissue structure, and the inflammatory response.

FIG. 12A, FIG. 12B, and FIG. 12C present the ELISA quantification results on Day 18 after wound creation in the skin of nude mice. FIG. 12A shows the expression of type I collagen; FIG. 12B shows the expression of type III collagen; FIG. 12C shows the ratio between type I and type III collagen expression levels. Asterisks indicate p-values less than 0.05. The laser combined with PD group showed the highest expression of type I collagen, followed by the laser group, PEG 1000 group, and PD group. These findings indicate that treatment with picosecond laser in combination with PDs promotes the orderly and structured arrangement of collagen in newly formed tissue after wound repair and healing. Additionally, the laser combined with PD group and laser group exhibited the highest type I/type III collagen ratio (shown as FIG. 12C), suggesting a predominance of type I collagen and a reduction in type III collagen in the wound. Mature scars typically show a higher type I/type III collagen ratio, while immature or hypertrophic scars tend to have a lower ratio, likely because mature scars replace original tissue during the healing process, increasing the relative expression of type I collagen.

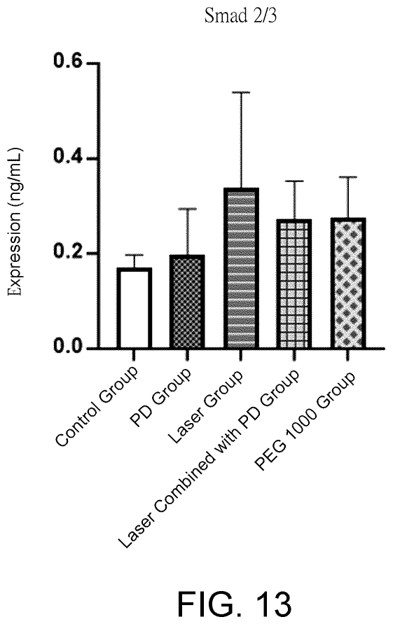

FIG. 13 shows the ELISA quantification results on Day 18 after wound creation in the skin of nude mice. The laser group and laser combined with PD group exhibited higher SMAD2/3 expression compared with other groups, with the laser combined with PD group showing the highest SMAD2/3 levels. These findings suggest that treatment with the combination of picosecond laser and PDs enhances SMAD2/3 expression, thereby regulating collagen production to improve wound repair and healing and improved wound healing outcomes.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A method of treating skin, which comprises administering to a subject in need thereof a composition, wherein the composition comprises a polymer dot.

2. The method of claim 1, further comprises topically administering to the subject a picosecond laser.

3. The method of claim 1, wherein the composition is configured to promote skin repair and/or skin regeneration of the subject.

4. The method of claim 1, wherein the composition is configured to enhance wound healing and/or inhibit scar formation on the skin of the subject.

5. The method of claim 1, wherein the polymer dot comprises a carbon dot.

6. The method of claim 5, wherein the carbon dot is a dendritic carbon dot.

7. The method of claim 6, wherein the dendritic carbon dot comprises a non-conjugated structure.

8. The method of claim 6, wherein the dendritic carbon dot comprises a first monomer of formula (I):
A₂B₃ (I), wherein A₂ is a dianhydride, and B₃ is a polyetheramine.

9. The method of claim 8, wherein the dianhydride comprises a bridged bicyclic olefinic structure or a non-olefinic structure.

10. The method of claim 8, wherein a terminal of the dendritic carbon dot comprises an amino group, and the first monomer is aliphatic.

11. The method of claim 6, wherein the dendritic carbon dot comprises a second monomer of formula (II):
AB₂ (II), wherein
A is an amino group and B₂ is a carboxylic acid; or
A is a carboxylic acid and B₂ is an amino group.

12. The method of claim 11, wherein
an interior of the dendritic carbon dot comprises an olefinic structure;
the dendritic carbon dot comprises an amino group structure; and
a terminal of the dendritic carbon dot comprises a carboxyl group structure; and the second monomer is aliphatic.

13. The method of claim 1, wherein the composition further comprises a humectant.

14. A method of treating skin, which comprises administering to a subject in need thereof a composition and a picosecond laser, wherein the composition comprises a polymer dot.

15. The method of claim 14, the composition is configured to enhance wound healing and/or inhibit scar formation on the skin of the subject.

16. The method of claim 14, wherein the polymer dot comprises a dendritic carbon dot.

17. The method of claim 16, wherein the dendritic carbon dot comprises:
a first monomer of the formula (I):
A₂B₃ (I), wherein A₂ is a dianhydride, and B₃ is a polyetheramine; and
a second monomer of formula (II):
AB₂ (II), wherein
A is an amino group and B₂ is a carboxylic acid; or
A is a carboxylic acid and B₂ is an amino group.

18. The method of claim 17, wherein
the composition further comprises an auxiliary media configured for irradiation by the picosecond laser;
the composition is configured to induce cell migration, cell proliferation, and/or anti-inflammatory response after irradiation by the picosecond laser; and
the composition and the picosecond laser are topically administering to the subject to enhance wound healing and/or tissue regeneration of the subject.

19. The method of claim 18, wherein
the composition further comprises a humectant containing polyethylene glycol with molecular weight of 1000 g/mol to 2000 g/mol;
the dendritic carbon dot has a concentration of 1 mg/ml to 10 mg/ml; and
the subject is administered with 100 to 1000 pulses of the picosecond laser having output energy of 0.25 J/cm² to 1.50 J/cm².

20. Use of a polymer dot for manufacture of a composition for treating skin of a subject in need, wherein the polymer dot comprises a carbon dot and the composition is configured to promote skin repair and/or skin regeneration of the subject, or enhance wound healing and/or inhibit scar formation on the skin of the subject.
